(19) United States
(12) Patent Application Publication
(10) Pub. No.: US 2003/0228271 A1
(43) Pub. Date: Dec. 11, 2003

(54) High-concentration aqueous dispersions comprising hydrophobic microfine metal oxide particles and dispersion auxiliaries

(75) Inventors: Christoph Batz-Sohn, Hanau (DE); Petra Brandt, Duisburg (DE); Thomas Dietz, Essen (DE); Steffen Hasenzahl, Hanau (DE); Klaus Jenni, Essen (DE); Kathrin Lehmann, Leverkusen (DE); Ralf Mathiak, Gladbeck (DE); Angela Ruttgerodt, Leverkusen (DE)

Correspondence Address: SCULLY SCOTT MURPHY & PRESSER, PC, 400 GARDEN CITY PLAZA, GARDEN CITY, NY, 11530

(73) Assignees: Goldschmidt AG, Essen (DE); Degussa AG, Dusseldorf (DE)

(21) Appl. No.: 10/456,277

(22) Filed: Jun. 6, 2003

(30) Foreign Application Priority Data
Jun. 6, 2002 (DE) 102 25 122.3

(51) Int. Cl. A61K 7/075; A61K 7/08; A61K 9/14

(52) U.S. Cl. 424/70.23

(57) ABSTRACT

Aqueous dispersions comprising:

A) hydrophobically coated microfine metal oxide particles; and

B) at least one of the compounds of the general formula (I)

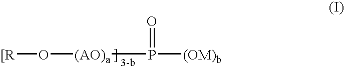

and optionally

C) at least one of the compounds of the general formula (II)

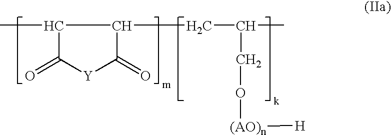

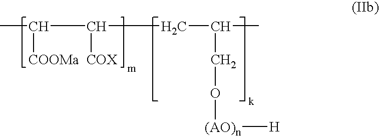

and optionally

D) further auxiliaries and additives,

E) water.

### DESCRIPTION

1. Field of the Invention

The present invention relates to aqueous dispersions comprising hydrophobic microfine metal oxide particles and, as dispersion auxiliaries, a combination of phosphate esters and maleic anhydride-acrylate copolymers. The present invention also relates to the use of these dispersions for the preparation of cosmetic formulations, in particular sunscreen formulations.

2. Background of the Invention

To protect skin against over-intensive UV radiation, cosmetic preparations, such as creams or lotions, containing UV filters are used which are largely transparent and pleasant to use on the skin.

UV filters comprise one or more organic compounds which absorb in the wavelength range between 290 and 400 nm: UVB radiation (290 to 320 nm); UVA radiation (320 to 400 nm).

The higher-energy UVB radiation causes typical sunburn symptoms and is also responsible for suppressing the immune defence, while UVA radiation, which penetrates more deeply into the layers of skin, causes premature aging of the skin. Since the combined effect of the two types of radiation is said to favor the formation of light-induced skin cancer diseases such as skin cancer, the search for ways of significantly improving the UV protection has been ongoing for many years.

It has been found that microfine (ultrafine) pigments based on metal oxides can also scatter, reflect and absorb UV radiation. Highly-dispersed formulations containing microfine pigments based on metal oxides represent an effective addition to organic UV filters in sunscreen compositions.

Microfine titanium dioxide is used widely in cosmetic formulations since it is chemically inert, toxicologically safe and leads neither to skin irritations nor to sensitization. Microfine titanium dioxide is the currently most used and most important mineral light protection substance. In addition to titanium dioxide, microfine zinc oxide is used to an increasing degree.

A distinction is made between coarsely divided material (pigment) and finely divided material (micropigment). For the micropigments, the average primary particle size is usually significantly less than 200 nm, mostly in the range from 10 to 100 nm, usually less than 50 nm.

The coarsely divided pigment (0.2 to 0.5 μm) absorbs or reflects broadly and relatively consistently over the entire UV region and the visible light region, while the finely divided material exhibits a significant increase in activity in the UV region with a simultaneous loss of activity in the long-wave UVA and, in particular in the visible, region. Since only a little visible light is reflected, preparations based on this active ingredient are largely transparent.

Due to their particularly large specific surface areas, the micro fine TiO₂ particles are photoactive and are able to generate reactive species (e.g. hydroxyl radicals). For use in cosmetic compositions, it is therefore necessary to suppress the photochemical activity of the microfine TiO₂ particles. This is achieved by inorganic and organic surface components, such as, for example, Al₂O₃, SiO₂ and/or fatty acid (salts), and siloxanes. These substances can adhere to the surface by chemisorption or physisorption (lattice doping/coating). Chemisorption leads to grades which are suitable for cosmetic light protection agents.

The primary particles of microfine titanium dioxide are not present in the dry pigment powder in isolated form, but rather form aggregates and agglomerates.

Primary particles refer to the smallest particles which are formed during the preparation of the pigments. Primary particles can be in the form of individual crystallites or else in the form of two or more crystallites which have intergrown tightly with one another along faces. Aggregates refer to particles composed of two or more primary particles, in which the primary particles are intergrown with one another along faces. Agglomerate is understood as meaning an association of primary particles or aggregates that are held together via attractive forces, such as, for example, hydrogen bridge bonds.

Agglomerates are present in every pigment powder, but are undesired in cosmetic transparent formulations since they can be identified as particles on the skin, often times with the naked eye. Moreover, agglomerates in cosmetic formulations reduce the transparency of the formulation as well as the UV protective action of a sunscreen composition and settle out during storage. Agglomerates therefore have to be largely comminuted again.

The entire process of incorporation, comminution and simultaneous distribution of solids in a liquid phase is referred to as dispersion.

As the primary particle size decreases, the specific surface area increases, as does the active area for the formation of aggregates and agglomerates, and also for adsorption processes. A result of the foregoing is that the stability of the emulsion can be endangered.

The comminution of the agglomerates and wetting of the newly provided surfaces is only possible with the aid of high shear forces and is carried out in practice in a large number of different special machines, such as, in particular, dissolvers and ball mills.

In practice, it has been found that as the finely divided nature of the particles increases, so too do the dispersion problems, with the result that the dispersion process overall represents one of the most complex sub-steps in the preparation of cosmetic formulations.

The requirements of practice therefore involve separating the most complex part of the dispersion—the comminution of the agglomerates—from the preparation of the actual cosmetic formulations, and preparing stable aqueous dispersions with the highest possible content of microfine TiO₂ which preferably have a low-viscosity or at least are pumpable or flowable.

A large number of proposals have been made which aim to solve this problem.

British Patent GB-A-2 206 339 describes dispersions of titanium dioxide particles of particle size from 0.01 to 0.15μ in organic oils and dispersion auxiliaries based on polyesters, salts of hydroxycarboxylic acids and/or hydroxyl-group-free C₆₋₂₂-fatty acids or salts thereof, as well as the use thereof as sunscreens.

WO-A-90/06103 proposes to reduce the clumping tendency (tendency for reagglomeration of titanium dioxide particles with particle sizes <100 nm) through coatings made of phospholipids.

DE-A-39 41 543 describes a process for the preparation of aqueous dispersions of needle-like finely divided titanium dioxide that is optionally coated with hydrous metal oxides, by grinding the titanium dioxide particles in the presence of a polycarboxylic acid or salt thereof as dispersant, and the use as sunscreens.

Although these dispersions have tendential improvements, the prior art dispersions still have the disadvantage that the aqueous dispersions comprise insufficiently high contents of microfine TiO₂, sediment during storage and/or the photoactivity is still too high.

A further significant disadvantage is that the prior art dispersions are not stable in the pH range from about 5 to 7 (i.e., the pH of the surface of skin) which is particularly preferred for cosmetic formulations.

An object of the present invention is therefore to overcome the existing disadvantages and to prepare stable high-concentration, aqueous dispersions of microfine metal oxide particles, in particular microfine titanium dioxide, with comparatively low viscosities, which are also stable in the acidic physiologically favorable pH range.

### SUMMARY OF THE INVENTION

The aforementioned object is achieved in the present invention through the use of hydrophobically coated microfine metal oxide particles and a combination of phosphate esters and maleic anhydride-acrylate copolymers as dispersion auxiliaries.

As well as having a low photocatalytic activity, hydrophobically coated titanium dioxide has a low tendency for reagglomeration since, in particular, the formation of hydrogen bridges via partially present Ti—OH groups of adjacent TiO₂ particles is not possible or is possible only to a low degree. Since hydrophobically coated titanium dioxides are usually not sufficiently water-wettable, aqueous dispersions of these substances are not hitherto known.

The present invention therefore provides aqueous dispersions comprising

A) hydrophobically coated microfine metal oxide particles and, as dispersion auxiliaries,

B) at least one of the compounds of the general formula (I)

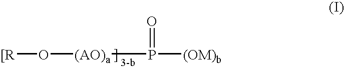

in which

R is an optionally branched alkyl radical having 6 to 22 carbon atoms which may or may not contain multiple bonds and may or may not contain hydroxyl groups;

A is an ethylene radical, a propylene radical, an isopropylene radical, or a butylene radical;

M is H, an ammonium ion or an alkali metal cation;

a is 0 to 30;

b is 0 to 2; and optionally

C) at least one of the compounds of the general formula (II)

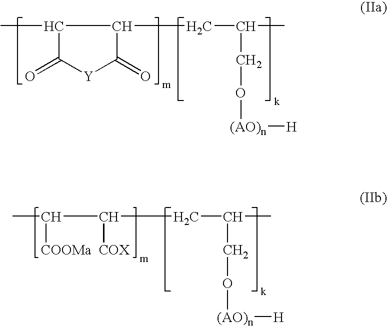

in which

M is hydrogen, a monovalent or divalent metal cation, an ammonium ion, or an organic amine radical;

a is 1, or where M is a divalent metal cation, a is 0.5;

X is —OMₐ or —O—(CₚH₂ₚO)_{q}—R¹ where R¹═H, an aliphatic hydrocarbon radical having 1-20 carbon atoms, a cycloaliphatic hydrocarbon radical having 5 to 8 carbon atoms, an optionally substituted aryl radical having 6 to 14 carbon atoms, p=2 to 4, q=0 to 100, —NHR² and/or —NR²₂ where R²═R¹ or —CO—NH₂;

Y is O,or NR²;

A¹ is an ethylene radical, a propylene radical, an isopropylene radical, or a butylene radical;

m is 10 to 30;

n is 0 to 50; and

k is 10 to 30, where the sum

m+k is in the range from 20 to 60, preferably from 20 to 40; optionally

D) further auxiliaries and additives; and

E) water.

The present invention further provides for the use of the aqueous dispersions described above for the preparation of the cosmetic formulations.

### BRIEF DESCRIPTION OF THE DRAWINGS

FIG. 1 is a plot of Zeta potential [mV] or specific conductivity [S/m] vs. pH for the formulation of Example 4 of the present invention.

FIG. 2 is a plot of Zeta potential [mV] or specific conductivity [S/m] vs. pH for the formulation of Example 8 of the present invention.

FIGS. 3A and 3B are plots of Zeta potential [mV] or specific conductivity [S/m] vs. pH for Market products I and II.

### DETAILED DESCRIPTION OF THE INVENTION

The phosphoric esters used according to the present invention are represented by the general formula (I)

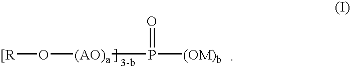

in which the variables R, A, a, b and M are as defined above.

The nature of the industrial preparation process results in mixtures in which the desired main component, according to the present invention preferably the monoester or the diester, are present predominantly along with small proportions of other possible reaction products.

The phosphoric esters are prepared by reacting fatty alcohols R—OH or fatty alcohol alkoxylates R—O—(AO)ₐ—H with phosphoric acid or derivatives thereof by known processes.

The co-used fatty alcohols can be prepared by known processes by reducing fatty acids or esters thereof in the presence of catalysts. In the direct hydrogenation, fatty alcohols from triglycerides are reacted with hydrogen in a single-stage process in a tubular reactor over a Cu/Cr catalyst, giving the reaction products fatty alcohols, 1,2-propanediol and water. In other processes, a fatty alcohol from triglycerides is prepared via a transesterification step with subsequent hydrogenation of the fatty acid ester.

The fatty acids co-used are, individually or in mixtures, fatty acids, such as caprylic acid, capric acid, 2-ethylhexanoic acid, lauric acid, myristic acid, palmitic acid, palmitoleic acid, isostearic acid, stearic acid, hydroxystearic acid, (ricinoleic acid), dihydroxystearic acid, oleic acid, linoleic acid, petroselic acid, elaidic acid, arachidic acid, behenic and erucic acid, gadoleic acid, and the technical-grade mixtures produced during the pressurized cleavage of natural fats and oils, such as oleic acid, linoleic acid, linolenic acid, and in particular rapeseed oil fatty acid, soybean oil fatty acid, sunflower oil fatty acid, and tall oil fatty acid. In principle, all fatty acids with a similar chain distribution are suitable for use in the present invention.

The content of unsaturated fractions in these fatty acids or fatty acid esters is, where necessary, adjusted to a desired iodine number by known catalytic hydrogenation processes, or is achieved by mixing completely hydrogenated fatty components with nonhydrogenated fatty components. The iodine number, being a measure of the average degree of saturation of a fatty acid, is the amount of iodine which is taken up by 100 g of the compound to saturate the double bonds.

Preference is given in the present invention to using alcohols from partially hydrogenated C_{8/18}-coconut or palm fatty acids, rapeseed oil fatty acids, sunflower oil fatty acids, soybean oil fatty acids and tall oil fatty acids, having iodine numbers in the range from about 80 to 150 and in particular from technical-grade C_{8/18}-coconut fatty acids, where, in some instances, a choice of cis/trans isomers, such as elaidic acid-rich C _{16/18}-fatty acid cuts, may be advantageous. The fatty acids are standard commercial products and are supplied by various companies under their respective trade names.

As well as the fatty alcohols, Guerbet alcohols and their alkoxylates can also be co-used.

The alcohol alkoxylates R—O—(AO)ₐ—H can be obtained by known processes by the addition reaction of alkylene oxides in the presence of acidic or basic catalysts. The radical -(AO)ₐ— here represents radicals such as ethylene oxide, propylene oxide, butylene oxide and/or tetrahydrofuran, preferably ethylene oxide, where a is an average value of up to 30, preferably 3 to 15 units.

In the general formula, -(AO)ₐ— means either a homopolymer of one of said alkylene oxides, or block copolymers or copolymers with random distribution of two or more of the monomers within the polymer molecule.

These products are commercially available. They are co-used in amounts of from 0.5 to 30%, based on aqueous dispersions, preferably from 3 to 15%, based on aqueous dispersions.

In the compounds of the general formula II, optionally co-used according to the present invention

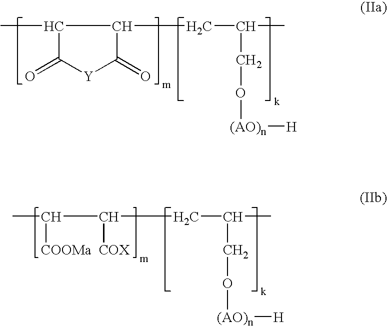

in which

M is hydrogen, a monovalent or divalent metal cation, an ammonium ion, or an organic amine radical;

a is 1, or where M is a divalent metal cation, a is 0.5;

X is —OMₐ or —O—(CₚH₂ₚO)_{q}—R¹ where R¹═H, an aliphatic hydrocarbon radical having 1-20 carbon atoms, a cycloaliphatic hydrocarbon radical having 5 to 8 carbon atoms, an optionally substituted aryl radical having 6 to 14 carbon atoms, p=2 to 4, q=0 to 100, —NHR² and/or —NR²₂ where R²═R¹ or CO—NH₂;

Y is O, or NR²;

A¹ is an ethylene radical, a propylene radical, an isopropylene radical, or a butylene radical;

m is 10 to 30;

n is 0 to 50; and

k 10 to 30, where the sum

m+k is in the range from 20 to 60, preferably from 20 to 40, -(AO)ₐ— is either a homopolymer of one of said alkylene oxides, or block copolymers or copolymers with random distribution of two or more of the monomers within the polymer molecule,

the units

[ ]ₘ and [ ]ₖ can likewise be present as block copolymers or copolymers with random distribution of two or more of the monomers within the polymer molecule.

The products are co-used in amounts up to 30% by weight, preferably in amounts from 1 to 15% by weight, based on the aqueous dispersions.

Microfine metal oxide particles which can be used in the present invention are, in principle, all metal oxides that are customary in the respective fields of use. The term “microfine” or “ultrafine” is used herein to denote primary particle sizes of, on average, <about 250 nm, preferably to about 100 nm and below. For use in cosmetic formulations, the choice is naturally limited to compounds that are safe from a toxicological and dermatological point of view, such as, cerium oxide, zinc oxide, iron oxide and, in particular, titanium dioxide.

The microfine metal oxide particles co-used according to the present invention are standard commercial products that are obtainable under the respective trade names, also with inorganic or organic coatings, such as, for example, Micro Titanium Dioxide MT-500B, MT-100 Z and MT-100 TV (all Tri-K-Tayca), UV-Titan M 160, M 262, X 161 (all Kemira), Eusolex T-2000 (Merck), Uvinul TiO₂ (BASF), titanium dioxide T-805 and titanium dioxide T-817 (both Degussa).

According to the present invention, preference is given to titanium dioxide having an average primary particle sizes of <about 250 nm, preferably <100 nm and in particular in the range from 10 to 100, which are coated with fatty acid (salts) and, in particular, alkylsilanes.

Titanium dioxide T 805 (Degussa) has proven particularly advantageous in the present invention. Titanium dioxide T 805 consists, in crystallographic terms, of about 80% anatase and about 20% rutile and has a primary particle size of about 21 nm and is coated with trialkoxyoctylsilane. Titanium dioxide T 805 is characterized has having reduced photoactivity, reduced surface activity, high cosmetic acceptance, and very good water resistance.

In addition to the above mentioned components, further auxiliaries and additives known in this field can be co-used as desired. Illustrative examples of such auxilaries and additives include, but are not limited to: ethanol, propanol, butanol, propylene glycol, butylene glycol, pentylene glycol, hexylene glycol, alkoxylates, glycol ethers, glycols, polyethylene glycols, polypropylene glycols, polybutylene glycols, glycerol ester ethoxylates, glycerol, polyglycerol, sorbitol, sucrose, fructose, galactose, mannose, polysorbate, starch, xanthan gum, carrageenan gum, cellulose derivatives, alginates, glycol esters, sorbitan esters, opacifiers, solubilizers, ethoxylated fatty alcohols, sodium chloride, sodium sulfate, magnesium sulfate, buffer systems, cholesterol, pantothenic acid, ascorbic acid, polyacrylic acids, and carbomers.

The dispersions according to the present invention are preferably used for the preparation of cosmetic formulations, such as foundation, colored powders, lipstick, hair colorants, day creams and, in particular, sunscreen preparations. The dispersions can be in the customary forms, such as, for example, W/O or O/W dispersions (emulsions), gels, creams, lotions, sprays.

The resulting dispersions of the present invention are characterized by a high finely divided nature of the dispersed solid, long-term storage stability, low viscosity and high photostability.

The viscosity is measured using a Brookfield RVT, spindle 5, in accordance with the manufacturer's instructions and is, at room temperature at 10 revolutions per minute (rpm), between 10 and 40,000 mPas.

The zeta potential can be used as a characteristic of the electrostatic stabilization of a dispersion.

The zeta potential is the outwardly effective potential of the particle and represents a measure of the electrostatic interaction between individual particles. Zeta potential plays a role in the stabilization of suspensions and, in particular, of dispersions with dispersed microfine particles. At a zeta potential value of <−20 mV or >+20 mV, there is strong repulsion between the particles, the dispersions remain stable. At values within this range, the repulsion becomes so low that the van der Waals' forces permit the formation of agglomerates, leading to undesired sedimentation of the particles.

The dispersions according to the present invention can be prepared by methods generally known in this field, the mixing devices used being automatic dispersers with toothed discs, bead mills, rotor-stator systems, or Scandex shakers.

In an expedient manner, the dispersion additives and optionally co-used polyols are introduced into water, and the pigment is sprinkled in with appropriate stirring. The predispersion obtained in this way is then finely dispersed. 20

The aqueous dispersions comprise:

5 to 80% by weight of component A), in particular 20 to 60%,

0.5 to 30% by weight of component B), in particular 3 to 15%,

0 to 30% by weight of component C), in particular 1 to 15%,

0 to 20% by weight of component D), in particular 1 to 10%, add 100% by weight of water.

Auxiliaries and additives which may be co-used are glycerol, propylene glycol, butylene glycol and higher glycol, polyglycerols, sorbitol and comparable sugar alcohols, and 0.1 to 0.5% of water-soluble or water-dispersible preservatives.

The following examples illustrate formulations of the present invention which are made using the method and components described herein above.

### EXAMPLES 1 to 4

| Example | 1 | 2 | 3 | 4 |
|---|---|---|---|---|
| TiO₂ (Degussa T 805) | 40.0% | 40.0% | 40.0% | 40.0% |
| Compound of the formula II | 7.0% | 6.0% | 5.0% | 8.0% |
| (MW 15 000) | | | | |
| Rewophat ® EAK 8190 | 5.0% | 6.0% | 7.0% | — |
| Compound of the formula I | | | | |
| Guerbet alcohol C12 EO-4 | — | — | — | 6.0% |
| phosphate | | | | |
| formula I, b = 1 to 2; M = H | | | | |
| Glycerol | 10.0% | 10.0% | 10.0% | 10.0% |
| Water | 38.0% | 38.0% | 38.0% | 36.0% |
| Viscosity (mPas) | 340 | 477 | 430 | 1 400 |

### EXAMPLES 5 to 9

| Example | 5 | 6 | 7 | 8 |
|---|---|---|---|---|
| UV-Titan M 160⁽¹⁾ | 40.0% | | | |
| UV-Titan M 262⁽²⁾ | — | 40% | 40% | 40% |
| Compound of the formula II | 0.5% | 11.8% | 0.5% | — |
| (MW 15,000) | | | | |
| Guerbet alcohol C₁₈ EO-4 | 15.5% | 0.5% | 11.5% | 14% |
| phosphate | | | | |
| formula I, b = 1 to 2; M = H | | | | |
| Glycerol | 10.0% | 10.0% | 10.0% | 10% |
| Water | 34.0% | 37.7% | 38.0% | 36% |
| Viscosity (mPas) | 1 200 | 600 | 100 | 360 |

⁽¹⁾Commercial product from Kemira, Finland; coated with stearic acid.
⁽²⁾Commercial product from Kemira, Finland; coated with dimethicone.

### Example 9

| Example | 9 |
|---|---|
| TiO₂ (Degussa T 805) | 40.0% |
| Compound of the formula II | 3.0% |
| (MW 15 000) | |
| Guerbet alcohol C18-4, EO-4 | 9.0% |
| phosphate | |
| formula I, b = 1 to 2; M = H | |
| Glycerol | 10.0% |
| Water | 38.0% |
| Viscosity (mPas) | 2 800 |

Determination of the zeta potential:

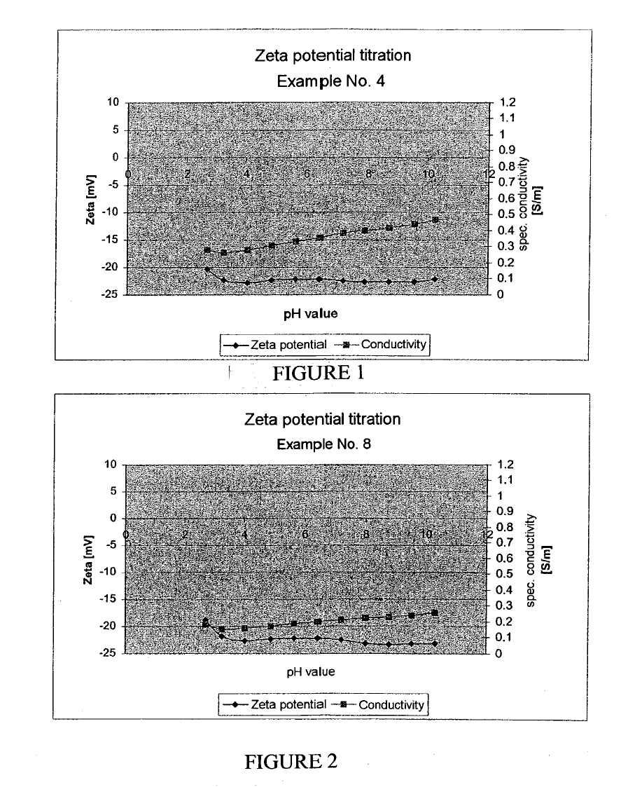

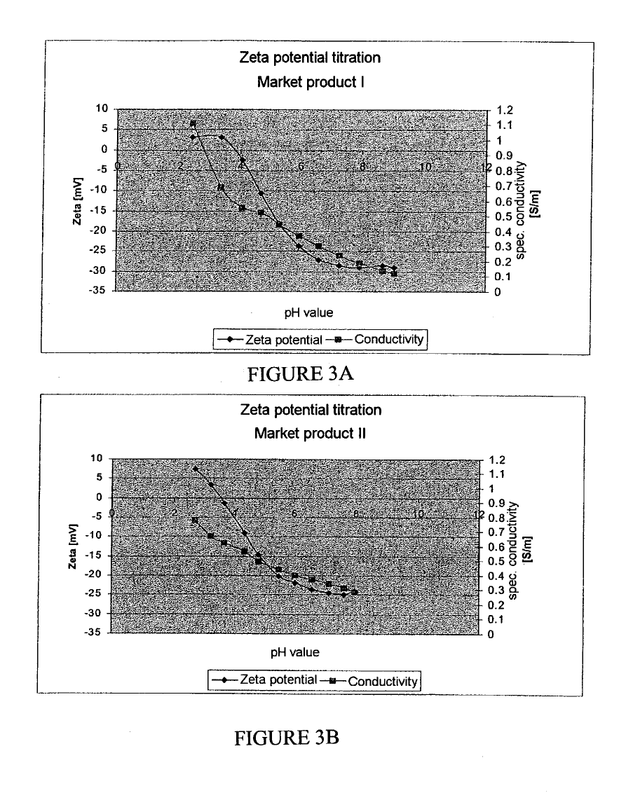

The zeta potential measurements for formulations of Example 4 and 8 were carried out using the DT-1200 instrument from Dispersion Technology, USA in accordance with the CVI (collodial vibration current) method. The results of the zeta potential measurements are shown in FIGS. 1 and 2, respectively. FIGS. 3A and 3B are plots of Zeta potential [mV] or specific conductivity [S/m] vs. pH for Market products I and II, respectively.

It is clear from the curves that the zeta potential of the dispersions according to the invention is <−20 mV in the entire pH range relevant for cosmetic formulations, and also beyond it, i.e. the dispersions are distinctly stable.

By contrast, standard commercial TiO₂ dispersions used for cosmetic formulations have an adequate zeta potential only in a relatively narrow pH range and achieve the isoelectric point in the cosmetically interesting pH range of about 3 to 5.

While the present invention has been particularly shown and described with respect to preferred embodiments thereof, it will be understood by those skilled in the art that the foregoing and other changes in form and detail may be made without departing from the spirit and scope of the present invention. It is therefore intended that the present invention not be limited to the exact forms and details described and illustrated, but fall within the scope of the appended claims.

What is claimed is:

1. An aqueous dispersion comprising:
A) hydrophobically coated microfine metal oxide particles; and
B) at least one of the compounds of the general formula (I) 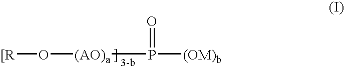
in which
R is an optionally branched alkyl radical having 6 to 22 carbon atoms which may or may not contain multiple bonds and may or may not contain hydroxyl groups;
A is an ethylene radical, a propylene radical, an isopropylene radical, or a butylene radical;
M is H, an ammonium ion or an alkali metal cation;
a is 0 to 30;and
b is 0 to 2.

2. The aqueous dispersion of claim 1 wherein R is a fatty alcohol radical having 6 to 22 carbon atoms, and a is a value between 1 and 30.

3. The aqueous dispersion of claim 1 wherein R is a Guerbet alcohol radical having 6 to 22 carbon atoms, and a is a value between 1 and 30.

4. The aqueous dispersion of claim 1 wherein the aqueous dispersion comprises 20 to 60% by weight of said microfine metal oxide particles.

5. The aqueous dispersion of claim 1 wherein the micro fine metal oxide particles are zinc oxide, titanium dioxide or a combination thereof that are hydrophobically coated with trialkoxyoctylsilane.

6. The aqueous dispersion of claim 1 wherein the micro fine metal oxides have a primary particle size of between 10 and 100 nm.

7. The aqueous dispersion of claim 1 further comprising
C) at least one of the compounds of the general formula (II) 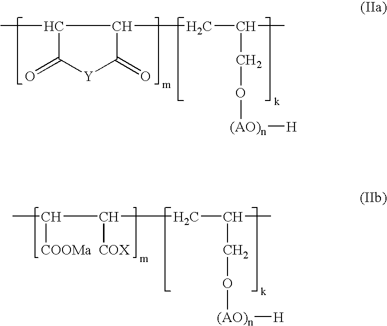
in which
M is hydrogen, a monovalent or divalent metal cation, an ammonium ion, or an organic amine radical;
a is 1, or where M is a divalent metal cation, a is 0.5;
X is —OMₐ or —O—(CₚH₂ₚO)_{q}—R¹ where R¹═H, an aliphatic hydrocarbon radical having 1-20 carbon atoms, a cycloaliphatic hydrocarbon radical having 5 to 8 carbon atoms, an optionally substituted aryl radical having 6 to 14 carbon atoms, p=2 to 4, q=0 to 100, —NHR² and/or —NR₂² where R²═R¹ or —CO—NH₂;
Y is O, or NR²
A¹ is an ethylene radical, a propylene radical, an isopropylene radical, or a butylene radical;
m is 10 to 30;
n is 0 to 50; and
k is 10 to 30, where the sum m+k is in the range from 20 to 60.

8. The aqueous dispersion of claim 7 wherein compounds of the general formula II in which A¹ is an ethylene radical, m is 10 to 30, n is 5 to 20, k is 10 to 30 and where the sum m+k is in the range from 20 to 40, are co-used as component C).

9. The aqueous dispersion of claim 7 wherein compounds of the general formula II in which R is an optionally branched alkyl radical having 8 to 18 carbon atoms which may or may not contain multiple bonds and may or may not contain hydroxyl groups, A is an ethylene radical, M=H or an alkali metal, a is 1 to 30, and b is 1 or 2, are co-used as component B).

10. The aqueous dispersion of claim 1 wherein the aqueous dispersion has a measured viscosity between 10 and 40,000 mPas.

11. The aqueous dispersion of claim 1 further comprising D) cosmetic auxilaries and additives; and E) water.

12. A cosmetic formulation comprising at least the aqueous dispersion of claim 1.
